(19) United States
(12) Patent Application Publication
(10) Pub. No.: US 2006/0122406 A1
(43) Pub. Date: Jun. 8, 2006

(54) Hydroxylation process for aromatic compounds containing a dioxa-heterocyclic system

(75) Inventors: Grazia Andreotti, Monza-Milan (IT); Pietro Panseri, Bergamo (IT); Vittorio Messori, Milan (IT); Oliviero Mariani, Madone-Bergamo (IT); Giezi Bognini, Ponte S.Pietro-Bergamo (IT)

Correspondence Address: OBLON, SPIVAK, MCCLELLAND, MAIER & NEUSTADT, P.C., 1940 DUKE STREET, ALEXANDRIA, VA, 22314, US

(73) Assignee: Borregaard Italia S.p.A., Milan (IT)

(21) Appl. No.: 10/521,452

(22) PCT Filed: Jul. 2, 2003
(86) PCT No.: PCT/EP03/07056

§ 371 Date: Sep. 21, 2005

(30) Foreign Application Priority Data
Jul. 26, 2002 (IT) MI2002A001662

(51) Int. Cl. C07D 319/14; C07D 323/02

(52) U.S. Cl. 549/362; 549/434

(57) ABSTRACT

Processs for the hydroxylation of aromatic compounds containing a dioxa-heterocyclic system which comprises directly hydroxlating said compounds with hydrogen peroxide in; the presence of a zeolitic catalyst having the general formula:
xTiO₂′(1−x)SiO₂ wherein x is i a number ranging from 0.0001 to 0.04.

The present invention relates to a hydroxylation process of aromatic compounds containing a heterocyclic system.

More specifically, the present invention relates to a process for the hydroxylation of 1,3-benzodioxoles.

Even more specifically, the present invention relates to a process for the oxidation of 1,3-benzodioxole (MDB) to give 5-hydroxy-1,3-benzodioxole (5-hydroxy-MDB).

Processes are known in literature, for the oxidation of aromatic compounds containing a heterocyclic system, such as benzodioxoles, which give, for example, products such as 5-hydroxy-MDB, a product which is used in agriculture and in the pharmaceutical and cosmetic industry and as an intermediate for organic syntheses.

English patent GB 2,323,843, for example, describes a process for the preparation of 5-hydroxy-MDB which consists in the oxidation of heliotropin with hydrogen peroxide (H₂O₂) in the presence of formic acid, according to the Baeyer Villiger reaction. This process, however, has the disadvantage of using a costly raw material as it is of a natural origin and is not easily available due to the fact that heliotropin is extracted from the sassafras plant currently protected by laws for the protection of the environment.

Another known process for producing 5-hydroxy-MDB is described in Japanese patent application 56-834865 of 1979. This process starts from MDB with the formation of the acetyl derivative, methylenedioxyacetophenone, followed by Baeyer Villiger oxidation using peracids. This method, currently in use, has the disadvantage of creating problems of an environmental nature due to the use of Friedel Kraft catalysts, the presence of methylenedioxyacetophenone and the use of dangerous oxidants, such as peracetic acid, in the oxidation phase of the corresponding acetophenone. As a whole, this synthesis method is characterized by two distinct phases, each of which is marked by heavy reaction conditions due to the use of particularly polluting catalysts and particularly dangerous reagents which are not easy to handle.

Another process described in Japanese patent application 63-45756 of 1993, starts from the derivative 5-bromo-1,3-benzodioxole followed by transformation into a borane derivative, by means of the Grignard reaction, and subsequent oxidation of the borane derivative with H₂O₂. This process is extremely lengthy, involves the use of various reagents which are not easy to handle on an industrial scale, such as the Grignard reactive intermediate and metallic magnesium, and the use of the borane agent which must then be oxidized in a second phase to the corresponding 5-hydroxy-MDB. The various passages of this process are onerous from an economical point of view due to the cost of the various reagents and waste load to be subjected to special disposal.

Another process, described in Japanese patent application 52-13384 of 1976, comprises the use of 1,2,4-triphenol as raw material and its subsequent cyclization with methylene chloride in a solvent medium, such as dimethylsulfoxide or dimethylformamide to obtain 5-hydroxy-MDB. This process has the disadvantage of the use of a raw material which is difficult to obtain and the drawback of providing very low reaction yields in the cyclization reaction, as the presence in the phenolic raw material of three equivalent hydroxyl groups leads to the formation of intermolecular methylene bridges in the reaction with the methylene chloride reagent. These by-products depress the yields, make it difficult to recover the desired product and must be sent for special combustion for their disposal.

In conclusion, none of the known processes for the production of 5-hydroxy-MDB has characteristics which make it interesting from an industrial point of view. These processes are, in fact, jeopardized by the use of costly raw materials, when they are not easily available, and also by the use of reagents which are difficult to handle or dangerous and in any case characterized by a heavy environmental impact.

The Applicant has now found that it is possible to overcome all the drawbacks of the processes of the known art for the oxidation of aromatic compounds containing a heterocyclic system, such as benzodioxoles, by means of a direct hydroxylation process of the substrate in the presence of a heterogeneous zeolitic catalyst and hydrogen peroxide.

An object of the present invention therefore relates to a process for the hydroxylation of aromatic compounds containing a heterocyclic system having general formula (I): 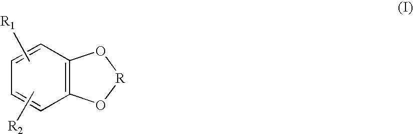
wherein R represents a C₁-C₄ (iso)alkylene radical, whereas R₁ and R₂, the same or different, represent a hydrogen atom or a CH₃ radical or a halogen, or a C₁-C₂ alkoxyl, which comprises directly hydroxylating said compounds having general formula (I) with H₂O₂ in the presence of a zeolitic catalyst having general formula (II):
xTiO₂·(1−x)SiO₂ (II)
wherein x is a number ranging from 0.0001 to 0.04, preferably from 0.01 to 0.025.

The zeolitic catalyst is known in literature as titanium-silicalite TS-1 and, more specifically, its preparation is described in U.S. Pat. No. 4,410,501 and in European patent 200,260. In the present invention the titanium-silicalite catalyst is used with a particle size ranging from 1 to 1000 μm, preferably from 5 to 100 μm or in the form of pellets.

According to the present invention, a substrate which is particularly suitable for being directly hydroxylated is that in which R is a methylene radical, whereas R₁ and R₂ are two hydrogen atoms. This product is 1,3-benzodioxole (MDB) and can be oxidized in position 5 to form 5-hydroxy-MDB.

The hydroxylation reaction can be effected in the presence of one or more solvents or directly in mass, by feeding H₂O₂, optionally diluted with H₂O, to a suspension of titanium-silicalite in the substrate, for example MDB, used in a large excess.

Solvents which can be used are:
products belonging to the group of aliphatic alcohols, in particular C₁-C₁₀ linear, branched or cyclic alcohols;
linear, branched or cyclic aliphatic ketones, with a number of carbon atoms ranging from 3 to 12;
linear, branched or cyclic saturated aliphatic hydrocarbons with a number of carbon atoms ranging from 5 to 12;
esters selected from dialkyl carbonates wherein the alkyl group contains from 1 to 4 carbon atoms, and esters of carboxylic acid having the formula CH₃—COO—R′ wherein R′ represents a C₁-C₄ radical;
linear, branched or cyclic aliphatic ethers, with a number of carbon atoms ranging from 3 to 12;
aliphatic nitrites having the formula R″−CN, wherein R″ represents a C₁-C₄ alkyl radical.

The heterogeneous titanium-silicalite catalyst, known in literature also as TS-1, can be used in batch reactions, in concentrations, with respect to the substrate, varying from 1 to 50% by weight, preferably from 5 to 30%. In this case, the hydrogen peroxide is fed onto a suspension of the TS-1 catalyst in the substrate or onto a suspension consisting of the TS-1 catalyst and a mixture of the substrate with a solvent possibly containing water.

Alternatively, the reaction can also be conveniently carried out in continuous by feeding hydrogen peroxide and the substrate on a layer of TS-1 catalyst or, preferably, by passing the reagents through a fixed bed of TS-1 catalyst in the form of pellets.

The hydrogen peroxide (H₂O₂) reagent can be used with the concentrations of commercial products, for example with concentrations in hydrogen peroxide ranging from 10 to 60% by weight, but can be further diluted to concentrations lower than 10%. In order to obtain high reaction yields, the molar ratio H₂O ₂/substrate can vary from 0.01 to 0.5, preferably from 0.1 to 0.3. The reaction can be carried out at a temperature ranging from 10 to 100° C., preferably from 40 to 80° C.

The process, object of the present invention, allows conversion selectivities of the substrate to hydroxylated product to be obtained, in particular of the MDB substrate to 5-hydroxy-MDB, of up to 80% and with conversions up to 30%. The excess substrate which has not reacted and the TS-1 catalyst can be separated from the reaction mixture and recycled for the subsequent syntheses.

Some illustrative but non-limiting examples are provided for a better understanding of the present invention and for its embodiment.

### EXAMPLE 1

30 g of TS-1 catalyst, 150 g of methylisobutylketone, 150 g of acetone and 150 g (1.23 moles) of MDB, are charged into a reactor equipped with a stirrer, thermometer, condenser and heating/cooling jacket. The resulting suspension is heated to 60° C., maintaining the mass under stirring. 84 g of H₂O₂ at 10% (0.25 moles) are then fed in about 4 hours, the temperature of the suspension being maintained at 60° C. The suspension is left under stirring at 60° C. for an hour and the organic phase is subsequently analyzed after separation from the TS-1 catalyst.

The following results are obtained from GLC analysis of the organic phase: MDB conversion=15%; selectivity to 5-hydroxy-MDB=62%.

### EXAMPLE 2

The same procedure is used as in Example 1 except that only 300 g of acetone are charged instead of the acetone/methylisobutylketone mixture.

The following results are obtained from GLC analysis of the organic phase: MDB conversion=12%; selectivity to 5-hydroxy-MDB=58%.

### EXAMPLE 3

The same procedure is used as in Example 1, except that 70 g of H₂O are charged instead of the mixture of solvents and 28 g of H₂O₂ at 30% (0.25 moles), are dosed.

The following results are obtained from GLC analysis of the organic phase: MDB conversion=10%; selectivity to 5-hydroxy-MDB=25%.

### EXAMPLE 4

The same procedure is used as in Example 1, except that 33 g of n-hexane are charged instead of acetone.

The following results are obtained from GLC analysis of the organic phase: MDB conversion=10%; selectivity to 5-hydroxy-MDB=70%.

### EXAMPLE 5

The same procedure is used as in Example 1, except that 150 g of dimethyl carbonate are charged instead of the acetone/methylisobutylketone mixture.

The following results are obtained from GLC analysis of the organic phase: MDB conversion=13%; selectivity to 5-hydroxy-MDB=52%.

### EXAMPLE 6

The same procedure is used as in Example 1, except that 150 g of acetonitrile are charged instead of the acetone/methylisobutylketone mixture.

The following results are obtained from GLC analysis of the organic phase: MDB conversion=10%; selectivity to 5-hydroxy-MDB=45%.

### EXAMPLE 7

The same procedure is used as in Example 1, except that 300 g of terbutanol are charged instead of the acetone/methylisobutylketone mixture.

The following results are obtained from GLC analysis of the organic phase: MDB conversion=15%; selectivity to 5-hydroxy-MDB=48%.

1. A process for the hydroxylation of an aromatic compound comprising a heterocyclic system represented by formula (I): 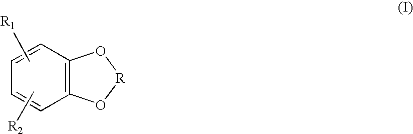
wherein R represents a C₁-C₄ (iso)alkylene radical, and R₁ and R₂ independently represent a hydrogen atom or a CH₃ radical, or a C₁-C₂ alkoxyl, said method comprising directly hydroxylating said compound represented by formula (I) with H₂O₂ in the presence of a zeolitic catalyst represented by formula (II):
xTiO₂·(1−x)SiO₂ (II)
wherein x is a number ranging from 0.0001 to 0.04.

2. The process according to claim 1, wherein the zeolitic catalyst is used with a particle size ranging from 1 to 1000 μm, or in the form of pellets.

3. The process according to claim 1, wherein R is a methylene radical and R₁ and R₂ are two hydrogen atoms.

4. The process according to claim 1, wherein the hydroxylation reaction is carried out in the presence of one or more solvents or directly in mass by feeding hydrogen peroxide, optionally diluted with H₂O, to a suspension of catalyst in the substrate.

5. The process according to claim 4, wherein the solvent is at least one solvent selected from the group consisting of:
aliphatic alcohols;
linear, branched or cyclic aliphatic ketones, with a number of carbon atoms ranging from 3 to 12;
linear, branched or cyclic saturated aliphatic hydrocarbons with a number of carbon atoms ranging from 5 to 12;
esters selected from dialkyl carbonates wherein the alkyl group comprises from 1 to 4 carbon atoms, and esters of carboxylic acid having the formula CH₃—COO—R′ wherein R′ represents a C₁ -C₄ radical;
linear, branched or cyclic aliphatic ethers, with a number of carbon atoms ranging from 3 to 12; and
aliphatic nitrites having the formula R″—CN, wherein R″ represents a C₁-C₄ alkyl radical.

6. The process according to claim 1, wherein the catalyst is used in batch reactions, in concentrations, with respect to the substrate, ranging from 1 to 50% by weight.

7. The process according to claim 1, wherein the reaction is carried out in continuous, feeding hydrogen peroxide and the substrate on a layer of catalyst or by passing the reagents through a fixed bed of catalyst in the form of pellets.

8. The process according to claim 1, wherein the H₂O₂ reagent is used in an aqueous solution with concentrations ranging from 1 to 60% by weight.

9. The process according to claim 1, wherein the molar ratio H₂O₂/substrate varies from 0.01 to 0.5.

10. The process according to claim 1, wherein the oxidation reaction is carried out at a temperature ranging from 10 to 100° C.
